(19) United States
(12) Patent Application Publication
(10) Pub. No.: US 2025/0281444 A1
(43) Pub. Date: Sep. 11, 2025

(54) COMPOUND WITH NEW SKELETON OF URSANE TRITERPENOID - ENT-KAURANE DITERPENOID DIMER, AND PREPARATION METHOD THEREOF AND APPLICATION THEREOF

(71) Applicant: NANJING UNIVERSITY OF CHINESE MEDICINE, Nanjing (CN)

(72) Inventors: Jinao DUAN, Nanjing (CN); Ming ZHAO, Nanjing (CN); Hongkai BI, Nanjing (CN); Junfei ZHOU, Nanjing (CN); Cai LU, Nanjing (CN); Chuanlin JIN, Nanjing (CN); Mengyu DAI, Nanjing (CN); Sheng GUO, Nanjing (CN); Shulan SU, Nanjing (CN); Hui YAN, Nanjing (CN)

(73) Assignee: NANJING UNIVERSITY OF CHINESE MEDICINE, Nanjing (CN)

(21) Appl. No.: 19/215,404

(22) Filed: May 22, 2025

(30) Foreign Application Priority Data
Mar. 31, 2025 (CN) 202510387829X

(51) Int. Cl. A61K 31/215; A61K 36/185; A61P 31/04; A61P 35/00; C07C 69/753

(52) U.S. Cl. CPC A61K 31/215; A61K 36/185; A61P 31/04; A61P 35/00; C07C 69/753; A61K 2236/15; A61K 2236/333

(57) ABSTRACT

Disclosed are two compounds with a new skeleton of ursane triterpenoid—ent-kaurane diterpenoid dimer and a preparation method thereof. According to the invention, chemical ingredients of a tuber of a West African plant I. trichantha are deeply studied, and separated to obtain two compounds Bisicacinol B (1) and Bisicacinol C (2), and the compounds are both a natural hybrid dimer formed by a DielsAlder addition or Michael addition reaction between a ursane triterpenoid with a unique spiro structure and an ent-kaurane diterpenoid. Pharmaceutical experiments show that the Bisicacinol B (1) and Bisicacinol C (2) prepared by the invention have a significant anti-tumor activity (on a pancreatic cancer, a colon cancer, a lung cancer, or a liver cancer), and can also be combined with an amphotericin B to exert a coordinated and synergistic anti-Candida albicans activity.

### CROSS-REFERENCE TO RELATED APPLICATIONS

This application claims priority to Chinese Patent Application Ser. No. CN202510387829X filed on 31 Mar. 2025.

### TECHNICAL FIELD

The present invention belongs to the field of medicine, and particularly to two compounds with a new skeleton of ursane triterpenoid-ent-kaurane diterpenoid dimer, and a preparation method thereof and an application thereof in an anti-tumor effect.

### BACKGROUND

I. trichantha is an endemic plant of cornus in Central and West Africa, and is a medicinal plant used by indigenous tribes in Nigeria and neighboring countries. This plant plays an important role in traditional medicine in Nigeria, and its tuber is used as a medicine by local people, which is widely used in the treatment of various diseases, such as poisoning, constipation, emesis, and malaria. The tuber of I. trichantha is rich in starch. During the famine years, local people ate the tuber after repeatedly washing it with water. Modern research shows that I. trichantha has many physiological activities, such as blood glucose decrease, an anti-convulsion effect, sedation, analgesia, and bacteriostasis. At present, there are few studies on chemical ingredients of I. trichantha.

### SUMMARY

The present invention aims to deeply study active ingredients of tuber of I. trichantha, and the active ingredients with potential activity are extracted and separated to provide scientific basis for clinical application.

Technical solution: according to the present invention, two compounds with a new skeleton of ursane triterpenoid-ent-kaurane diterpenoid dimer are separated from the tuber of I. trichantha, and the compounds are named Bisicacinol B (1) and Bisicacinol C (2).

Structural formulae of the compounds 1 and 2 of the present invention are as follows:

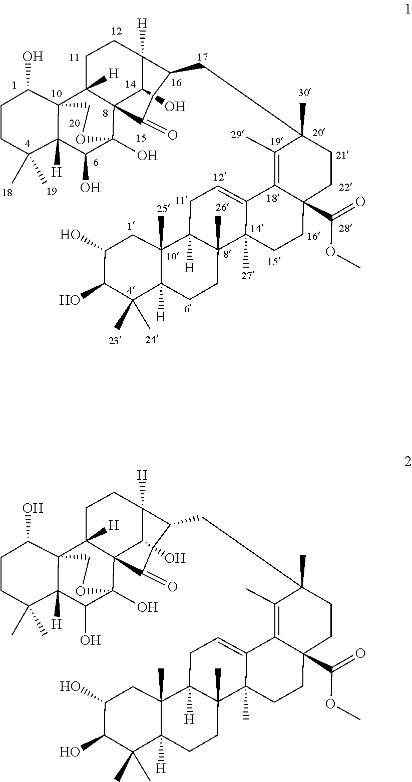

A preparation method of the compound with the new skeleton of ursane triterpenoid—ent-kaurane diterpenoid dimer according to the present invention comprises the following steps:

(1) weighing and crushing dried tuber of I. trichantha, adding ethanol aqueous solution, refluxing the mixture for extraction of medicinal material, filtering the mixture and then collecting a filtrate, and concentrating the filtrate under a reduced pressure until no ethanol smell exists to obtain a concentrated solution;

(2) extracting the concentrated solution obtained in the step (1) with petroleum ether, dichloromethane, ethyl acetate, and n-butanol respectively, and concentrating extracts under a reduced pressure to obtain a petroleum ether fraction, a dichloromethane fraction, an ethyl acetate fraction, an n-butanol fraction, and a raffinate fraction respectively;

(3) subjecting the dichloromethane fraction obtained in the step (2) to AB-8 macroporous resin atmospheric column chromatography, and eluting the product with ethanol aqueous solution to obtain 3 sub-fractions DCM-1 to DCM-3;

(4) subjecting the eluate fraction DCM-3 obtained in the step (3) to silica gel atmospheric column chromatography, and eluting the product with dichloromethane-methanol to obtain 7 secondary fractions DCM-3-1 to DCM-3-7; and

(5) subjecting the fraction DCM-3-5 obtained in the step (4) to medium-pressure preparative-MCI column chromatography, and gradiently eluting the product with methanol aqueous solution serving as a mobile phase to obtain 11 fractions DCM-3-5-1 to DCM-3-5-11; and subjecting the fraction DCM-3-5-9 to semi-preparative high-performance liquid chromatography to obtain the compounds 1 and 2.

As a preferred solution, the preparation method according to the present invention comprises the following steps:
(1) weighing and crushing dried tuber of I. trichantha, adding ethanol at a volume concentration of 80-95%, refluxing the mixture for extraction of medicinal material according to a material-liquid ratio of 1:6-20 for 1-3 times, for 1-3 hours each time, filtering the mixture and then collecting filtrates, combining the filtrates, and concentrating the combined filtrate under a reduced pressure until no ethanol smell exists to obtain an extractum;
(2) preparing the extractum obtained in the step (1) into a suspension with a proper amount of water, and then extracting the suspension with petroleum ether, dichloromethane, ethyl acetate, and n-butanol at an equal volume for 3-6 times respectively to obtain a petroleum ether fraction, a dichloromethane fraction, an ethyl acetate fraction, an n-butanol fraction, and a raffinate fraction respectively;
(3) subjecting the dichloromethane fraction obtained in the step (2) to macroporous resin sample stirring and then to macroporous resin atmospheric column chromatography, and gradiently eluting the product with ethanol aqueous solution serving as a mobile phase at volume ratios of 30:70, 60:40, 90:10, and 100:0 in sequence according to 4-6 column volumes in each gradient elution; and after thin-layer and ultra-high-performance liquid chromatography, concentrating and combining the products by a rotary evaporator to obtain 3 sub-fractions DCM-1 to DCM-3;
(4) subjecting the eluate fraction DCM-3 obtained in the step (3) to silica gel sample stirring and then to silica gel atmospheric column chromatography, and gradiently eluting the product with dichloromethane-methanol serving as a mobile phase at volume ratios of 100:0, 50:1, 30:1, 20:1, 10:1, 7:1, 5:1, 3:1, 2:1, 1:1, 1:3, 1:7, and 0:100 in sequence according to 3-6 column volumes in each gradient elution; and after thin-layer and ultra-high-performance liquid chromatography, concentrating and combining the products by a rotary evaporator to obtain 7 sub-fractions DCM-3-1 to DCM-3-7;
(5) subjecting the fraction DCM-3-5 obtained in the step (4) to MCI sample stirring and then to medium-pressure preparative-MCI column chromatography, and gradiently eluting the product with methanol aqueous solution serving as a mobile phase, wherein A is water, and B is methanol; and a gradient elution program is as follows: 0.01-20.00 minutes, 30%-30% B; 20.00-50.00 minutes, 40%-40% B; 50.00-80.00 minutes, 45%-45% B; 80.00-110.00 minutes, 50%-50% B; 110.00-140.00 minutes, 55%-55% B; 140.00-170.00 minutes, 60%-60% B; 170.00-200.00 minutes, 70%-70% B; 200.00-230.00 minutes, 85%-85% B; 230.00-270.00 minutes, 100%-100% B, an elution flow rate of 20 mL/min, and detection wavelengths of 256 nm and 310 nm; and after ultra-high-performance liquid chromatography, concentrating and combining the products to obtain 11 fractions DCM-3-5-1 to DCM-3-5-11; and
(6) subjecting the eluate fraction DCM-3-5-9 of 200-220 minutes obtained in the step (5) to semi-preparative high-performance liquid chromatography, isocratically eluting the product with pure water A-methanol B serving as a mobile phase, and separating the eluate to obtain the compound 1 and the compound 2.

As a preferred solution, chromatographic conditions of the semi-preparative high-performance liquid chromatography above are as follows: a chromatographic column model of Hedera ODS preparative chromatographic column (10 nm, 5 μm, 10×250 mm), a column pressure of high-performance liquid chromatography of 9.8 MPa, a column temperature of 22-26° C., an injection volume of 100 μL, a mobile phase of MeOH—H₂O (v/v, 80:20), a flow rate of 3 mL/min, and a detection wavelength of 310 nm. The monomer compounds Bisicacinol B (1) and Bisicacinol C (2) are obtained.

### Beneficial Effects:

The present invention deeply studies the chemical ingredients of the tuber of the plant I. trichantha in West Africa, which are separated to obtain the two compounds Bisicacinol B (1) and Bisicacinol C (2) with the new skeleton of ursane triterpenoid-ent-kaurane diterpenoid dimer. Pharmaceutical experiments show that the Bisicacinol B (1) and Bisicacinol C (2) prepared by the present invention have significant anti-tumor activity (comprising pancreatic cancer, colon cancer, lung cancer, or liver cancer), and good anti-bacterial activity (comprising Helicobacter pylori and Candida albicans).

According to the present invention, the compounds Bisicacinol B (1) and Bisicacinol C (2) obtained by extraction and separation may be prepared into various dosage forms with a pharmaceutically acceptable carrier.

### BRIEF DESCRIPTION OF THE DRAWINGS

FIG. 1 is a (+)-HR-ESI-MS graph of a compound 1;

FIG. 2 is a UV graph of the compound 1;

FIG. 3 is a ¹H NMR graph of the compound 1 (500 MHz, Methanol-d₄);

FIG. 4 is a ¹³C NMR graph of the compound 1 (125 MHz, Methanol-d₄);

FIG. 5 is a DEPT 135 graph of the compound 1 (125 MHz, Methanol-d₄);

FIG. 6 is a ¹H-¹H COSY graph of the compound 1 (500 MHZ, Methanol-d₄);

FIG. 7 is a HSQC graph of the compound 1 (¹H: 500 MHZ, ¹³C: 125 MHZ, Methanol-d₄);

FIG. 8 is a HMBC graph of the compound 1 (¹H: 500 MHZ, ¹³C: 125 MHZ, Methanol-d₄);

FIG. 9 is a NOSEY graph of the compound 1 (500 MHz, Methanol-d₄);

FIG. 10 is a (+)-HR-ESI-MS graph of a compound 2;

FIG. 11 is a UV graph of the compound 2;

FIG. 12 is a ¹H NMR graph of the compound 2 (500 MHZ, Methanol-d₄);

FIG. 13 is a ¹³C NMR graph of the compound 2 (125 MHz, Methanol-d₄);

FIG. 14 is a DEPT 135 graph of the compound 2 (125 MHz, Methanol-d₄);

FIG. 15 is a ¹H-¹H COSY graph of the compound 2 (500 MHZ, Methanol-d₄);

FIG. 16 is a HSQC graph of the compound 2 (¹H: 500 MHZ, ¹³C: 125 MHz, Methanol-d₄);

FIG. 17 is a HMBC graph of the compound 2 (¹H: 500 MHZ, ¹³C: 125 MHz, Methanol-d₄);

FIG. 18 is a NOSEY graph of the compound 2 (500 MHZ, Methanol-d₄);

FIG. 19 a correlation diagram of ¹H-¹H COSY, and key HMBC and NOESY of the compound 1;

FIG. 20 a correlation diagram of ¹H-¹H COSY, and key HMBC and NOESY of the compound 2; and

FIG. 21 is structural formulae of the compounds 1 and 2.

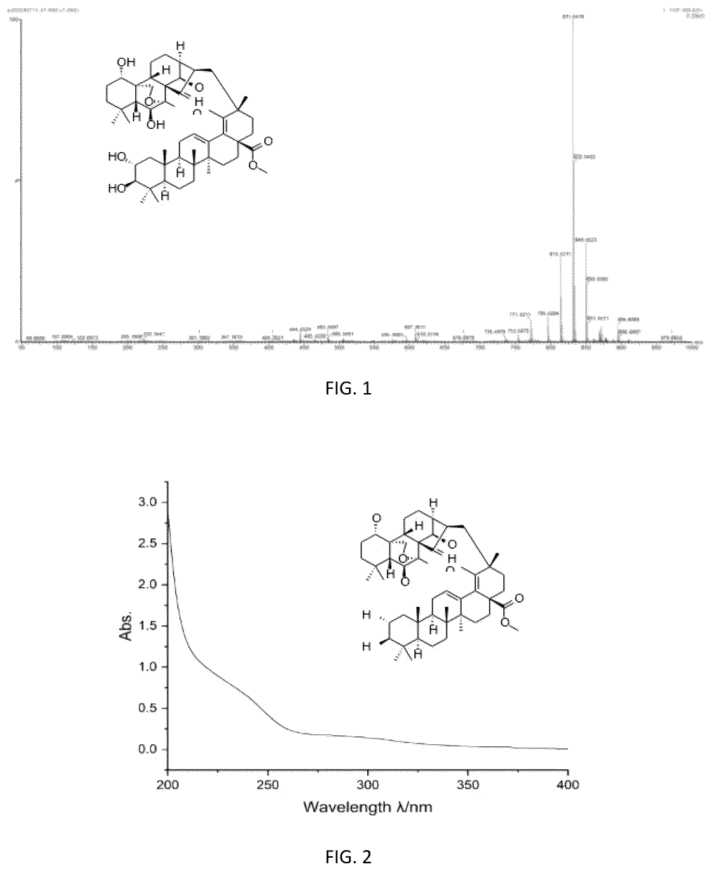

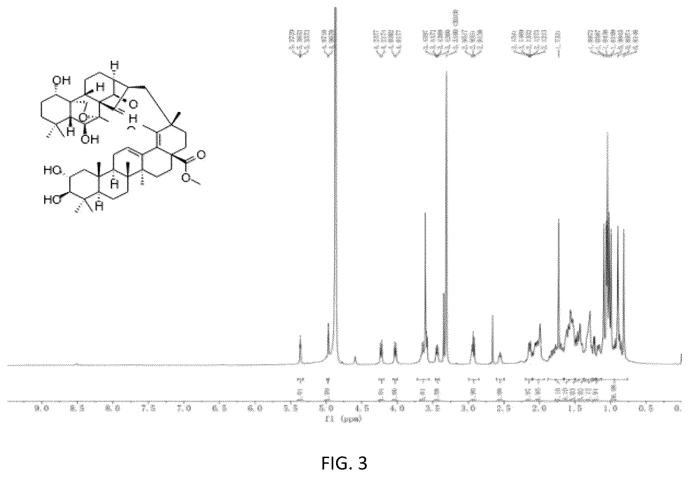

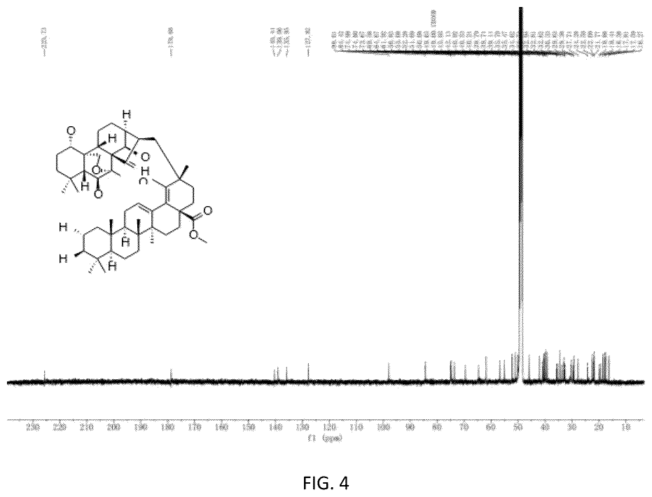

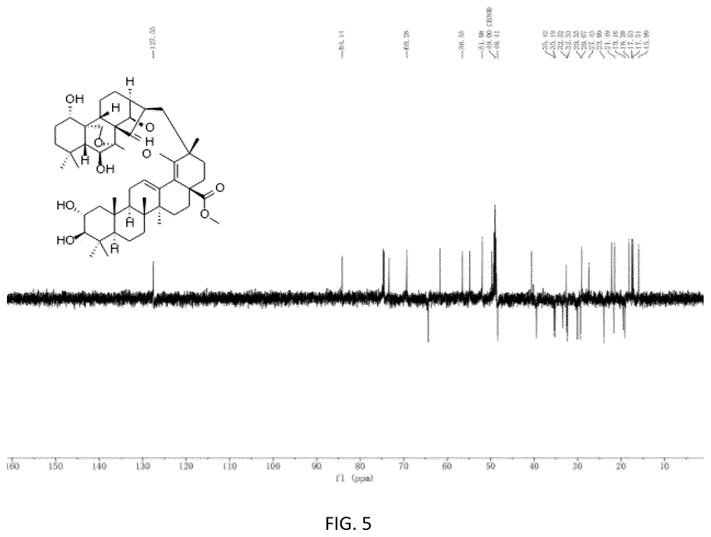

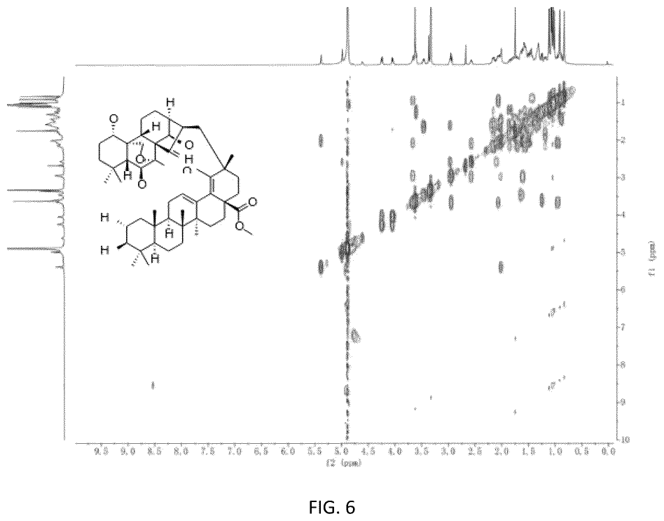

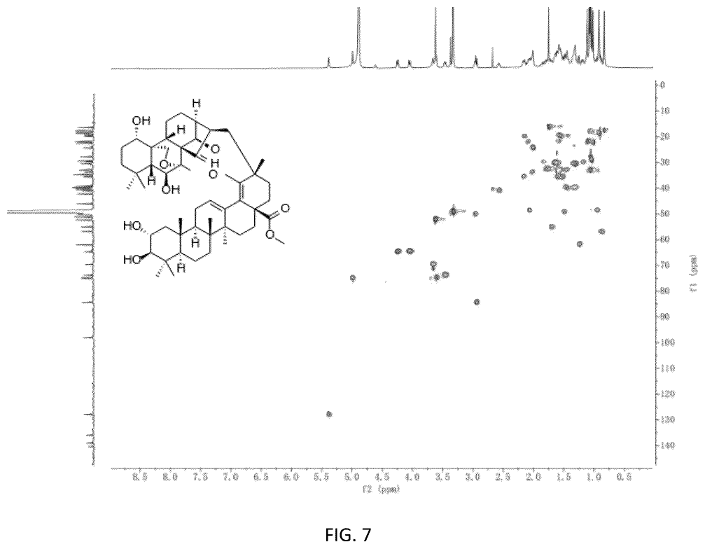

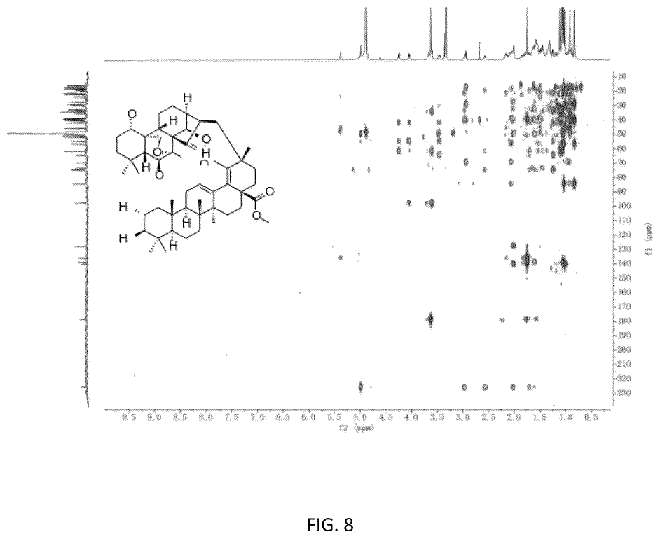

### DETAILED DESCRIPTION

### Embodiment 1

### 1. Instruments and Materials

### 1.1 Instruments

| Instrument | Manufacturer |
|---|---|
| ACQUITY UPLC high performance | Waters Company of America |
| liquid phase | |
| CHEETAH ®MP medium-pressure | Tianjin Agela Technologies |
| preparative chromatography | Co., Ltd. |
| Nuclear magnetic resonance | Bruker Company of Germany |
| instrument Bruker AV-500 | |
| Shimadzu LC-20 AR preparative | Shimadzu Company of Japan |
| high performance liquid | |
| chromatographic instrument | |
| R-300 rotary evaporator | BUCHI Limited Company of |
| | Switzerland |
| Intelligent artificial climate | Ningbo Saifu Experimental |
| box PRX-150B | Instrument Co., Ltd. |
| Quitix 125 D-1CN electronic scale | Sartorius Scientific |
| | Instruments Co., Ltd. |
| Milli-Q Advantage system ultrapure | Millipore Company of |
| water instrument | America |
| 1300 series A2 clean bench | Thermo Company of America |
| EnSpire multifunctional microplate | PerkinElmer Company of |
| reader | America |
| S-B50L vertical pressure steam | Jiangsu Jintan Medical |
| sterilizer | Instrument Factory |
| Adjustable pipette | Eppendorf Company of America |
| Microporous plate thermostatic | Hangzhou Miu Instruments |
| oscillator | Co., Ltd. |
| 7500 Real Time PCR System | Applied Biosystems Company of |
| | America |
| IS-RDV1 constant temperature | Crystal Technology & |
| oscillator | Industries, Inc. of America |
| PrimoStar inverted microscope | ZEISS Company of Germany |
| SIM-F140AY65-PC ice machine | Panasonic Co., Ltd. of Japan |
| Forma series II water jacket | Thermo Company of America |
| CO2 incubator | |
| TDL-80-2B centrifugal machine | Shanghai Anting Co., Ltd. |

### 1.2 Experimental Materials

Hedera ODS preparative chromatographic column (10 nm, 5 μm, 10 mm×250 mm); Waters ACQUITY UPLC BEH C₁₈ (2.1 mm×100 mm, 1.7 μm) chromatographic column; MCI GEL (CHP20, 75 μm-150 μm), and column chromatography silica gel (200-300 meshes); chromatographic acetonitrile, methanol and formic acid purchased from Merck Company of America; and analytical petroleum ether and ethyl acetate purchased from Nanjing

### Wanqing Chemical Reagent Co., Ltd.

A preparation method of a compound was implemented by the following steps.

(1) Dried tuber of I. trichantha was weighed and crushed, and added with ethanol aqueous solution at a volume concentration of 95%, the mixture was refluxed for extraction of medicinal material according to a material-liquid ratio of 1:10 for 3 times, for 2.5 hours each time, and filtered, then filtrates were collected and combined, and the combined filtrate was concentrated under a reduced pressure until no ethanol smell existed to obtain an extractum.

(2) The extractum obtained in the step (1) was prepared into a suspension with a proper amount of water, and then the suspension was extracted with petroleum ether, dichloromethane, ethyl acetate, and n-butanol at an equal volume for 5 times respectively to obtain a petroleum ether fraction, a dichloromethane fraction, an ethyl acetate fraction, an n-butanol fraction, and a raffinate fraction respectively.

(3) The dichloromethane fraction obtained in the step (2) was subjected to sample stirring according to sample: macroporous resin=1:1.5, and then to macroporous resin atmospheric column chromatography, and the product was gradiently eluted with ethanol aqueous solution serving as a mobile phase at volume ratios of 30:70, 60:40, 90:10, and 100:0 in sequence according to 5 column volumes in each gradient elution; and after thin-layer and ultra-high-performance liquid chromatography, the products were concentrated and combined by a rotary evaporator to obtain 3 sub-fractions DCM-1 to DCM-3.

(4) The eluate fraction DCM-3 obtained in the step (3) was subjected to sample stirring according to sample: silica gel=1:1.5, and then to silica gel atmospheric column chromatography, and the product was gradiently eluted with dichloromethane-methanol serving as a mobile phase at volume ratios of 100:0, 50:1, 30:1, 20:1, 10:1, 7:1, 5:1, 3:1, 2:1, 1:1, 1:3, 1:7, and 0:100 in sequence according to 4 column volumes in each gradient elution; and after thin-layer and ultra-high-performance liquid chromatography, the products were concentrated and combined by a rotary evaporator to obtain 7 sub-fractions DCM-3-1 to DCM-3-7.

(5) The fraction DCM-3-5 obtained in the step (4) was subjected to sample stirring according to sample: MCI=1:1.2, and then to medium-pressure preparative-MCI column chromatography, and the product was gradiently eluted with methanol aqueous solution serving as a mobile phase, wherein A was water, and B was methanol; and a gradient elution program was as follows: 0.01-20.00 minutes, 30%-30% B; 20.00-50.00 minutes, 40%-40% B; 50.00-80.00 minutes, 45%-45% B; 80.00-110.00 minutes, 50%-50% B; 110.00-140.00 minutes, 55%-55% B; 140.00-170.00 minutes, 60%-60% B; 170.00-200.00 minutes, 70%-70% B; 200.00-230.00 minutes, 85%-85% B; 230.00-270.00 minutes, 100%-100% B, an elution flow rate of 20 mL/min, and detection wavelengths of 256 nm and 310 nm; and after ultra-high-performance liquid chromatography, the products were concentrated and combined to obtain 11 fractions DCM-3-5-1 to DCM-3-5-11.

(6) The eluate DCM-3-5-9 of 200-220 minutes obtained in the step (5) was subjected to semi-preparative high-performance liquid chromatography, the product was isocratically eluted with pure water (A)-methanol (B) serving as a mobile phase (MeOH—H₂O, v/v, 80:20), the monomer compound 1 was collected in a time period of 52-57 minutes, and the monomer compound 2 was collected in a time period of 58-61 minutes.

### 3. Structural Analysis of Compounds

### 3.1 Structural Identification of Bisicacinol B (1)

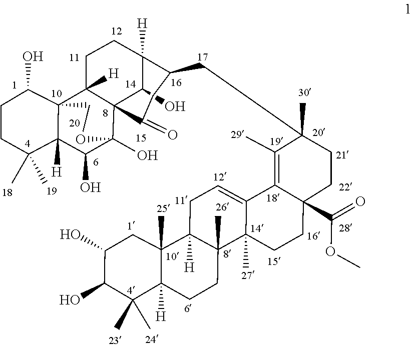

The compound 1 was white amorphous powder, [a]_{D}²⁰+15.0 (c 0.01, MeOH). In combination with ¹³C NMR data and a quasi-molecular ion peak m/z 849.5523 ([M+H]+, calculated value of C51H77010+ of 849.5517) given by a high-resolution mass spectrum (HRESIMS), it could be inferred that a molecular formula of the compound was C₅₁H₇₆O₁₀, and a degree of unsaturation was 14. A hydrogen spectrum of the compound 1 (Table 1) showed that the compound had 9 methyl proton signals δ_{H} (1.10, s, CH₃-18; 1.04, s, CH₃-19; 1.02, s, CH₃-23′; 0.81, s, CH₃-24′; 1.06, s, CH₃-25′; 0.90, s, CH₃-26′; 0.99, s, CH₃-27′; 1.73, s, CH₃-29′; 1.04, s, CH₃₋₃₀′); and 1 methoxy proton signal δ_{H} (3.62, s, OCH₃-28′). A ¹³C NMR spectrum and a DEPT spectrum showed that the compound had 51 carbon signals, comprising 9 methyl carbons, 14 methylene carbons (comprising 1 oxygen-bond methylene), 12 methine carbons (comprising 5 oxygen-bond methines), 13 non-hydrogen-bond carbons (comprising 1 oxygen-bond carbon), and 2 carbonyl carbons. In combination with a carbon spectrum of the compound 1 (Table 1), a DEPT spectrum and a HSQC spectrum showed that the compound 1 had 51 carbon signals, which could be classified as 9 methyl carbons δ_{C} 21.8 (CH₃-18), 32.9 (CH₃-19), 29.4 (CH₃-23′), 17.6 (CH₃-24′), 17.8 (CH₃-25′), 18.6 (CH₃-26′), 22.5 (CH₃-27′), 16.3 (CH₃-29′), and 27.7 (CH₃₋₃₀′); 1 methoxy carbon δ_{C} 52.3 (OCH₃-28); 14 methylene carbons (comprising 1 oxygen-bond methylene) δ_{C} 30.3 (C-2), 39.8 (C-3), 19.9 (C-11), 22.0 (C-12), 33.7 (C-17), 64.7 (C-20), 48.7 (C-1′), 19.4 (C-6′), 35.7 (C-7′), 39.1 (C-11′), 29.6 (C-15′), 32.6 (C-16′), 32.8 (C-21′), and 35.5 (C-22′); 12 methine carbons (comprising 5 oxygen-bond methines) δ_{C} 73.7 (C-1), 61.9 (C-5), 74.8 (C-6), 55.1 (C-9), 40.9 (C-13), 75.0 (C-14), 50.1 (C-16), 69.6 (C-2′), 84.4 (C-3′), 56.8 (C-5′), 49.4 (C-9′), and 127.8 (C-12′); 13 non-hydrogen-bond carbons (comprising 1 oxygen-bond carbon) dc 34.6 (C-4), 98.0 (C-7), 49.6 (C-8), 42.1 (C-10), 40.2 (C-4′), 39.7 (C-8′), 39.1 (C-10′), 140.4 (C-13′), 46.0 (C-14′), 51.1 (C-17′), 136.0 (C-18′), 139.1 (C-19′), and 40.5 (C-20′); and 2 carbonyl carbons δ_{C} 225.7 (C-15) and 178.7 (C-28′) respectively,

NMR data of unit I in the compound 1 were highly similar to those of oridonin, and the difference lied in a methine (δ_{C} 50.1, C-16) and a methylene (δ_{C} 33.7, C-17) of the unit I instead of an exocyclic double bond (δ_{C} 153.4, C-16; 120.6, C-17); and NMR data of unit II in the compound 1 were highly similar to those of goreishic acid, and the difference lied in that there was a methoxy (δ_{H} 3.62, δ_{C} 52.3, OCH₃-28′) in the unit II, and C-20 was a non-hydrogen-bond carbon (δ_{C} 40.5, C-20′) instead of a methine (δ_{H} 2.28, m; δ_{C} 24.8, C-20′). In a HMBC spectrum, related signals between CH-16 and C-20′ and between CH₂-17 and C-19′/C-20′/CH₂-21′/CH₃-30′ proved that the units I and II were connected by C-17 and C-20′ via a single bond.

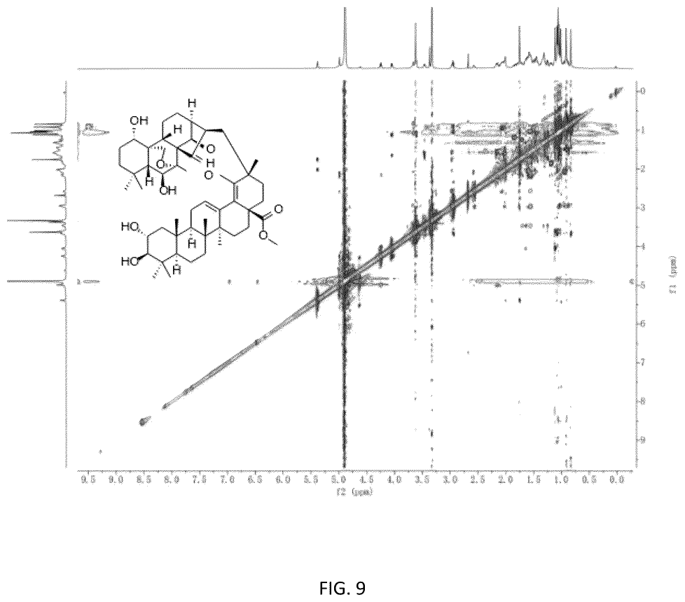

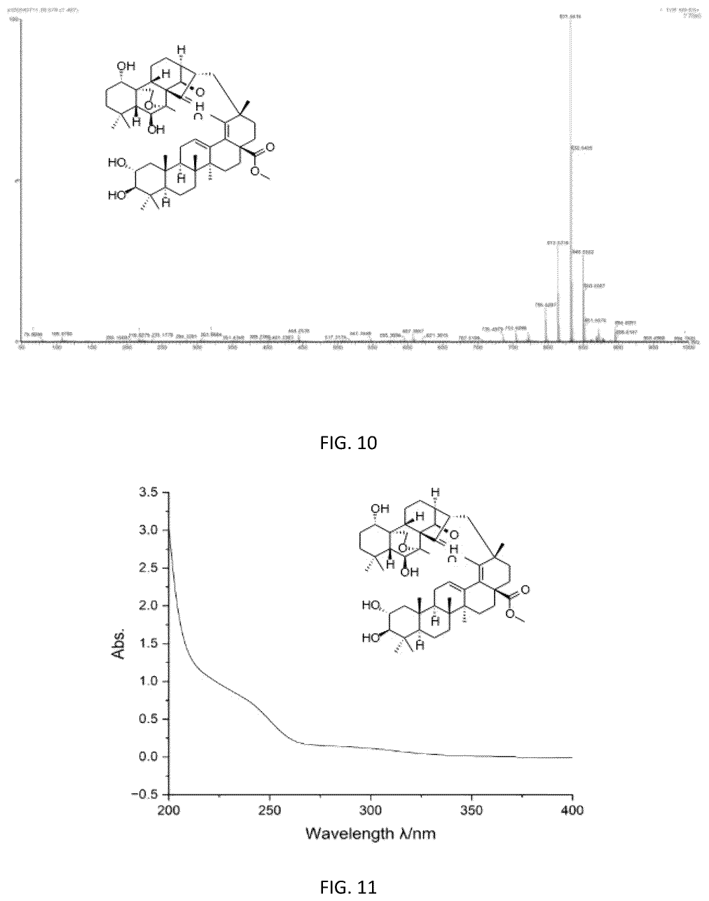

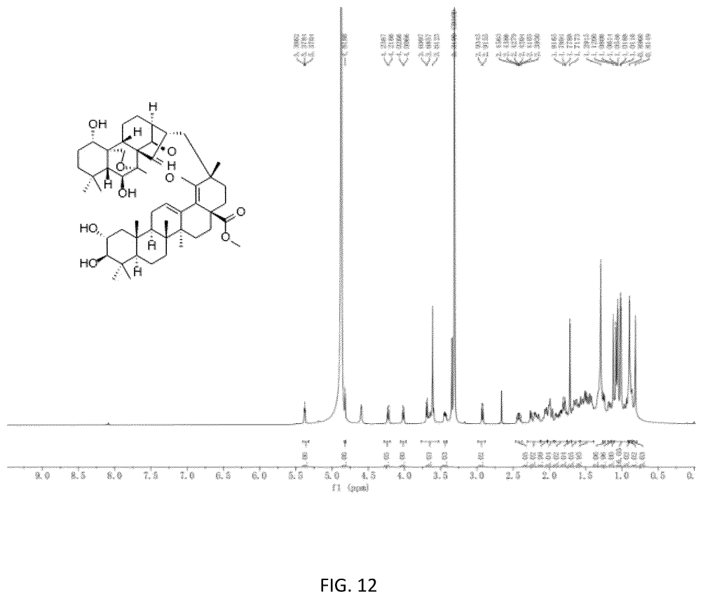

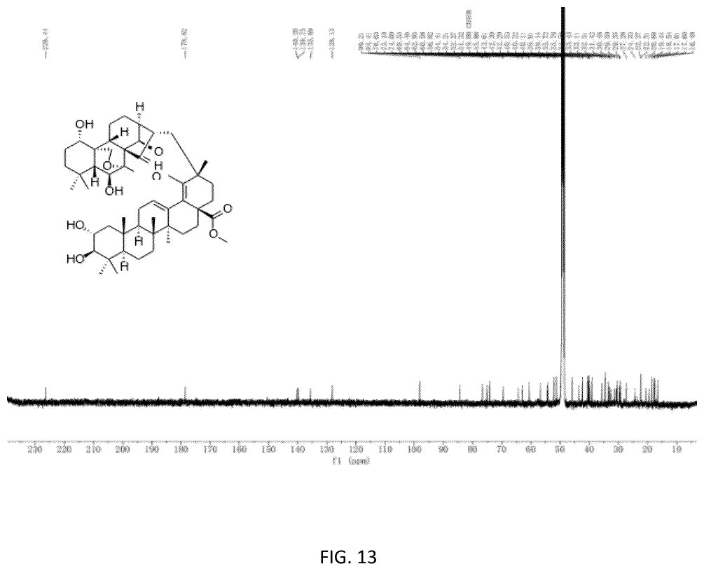

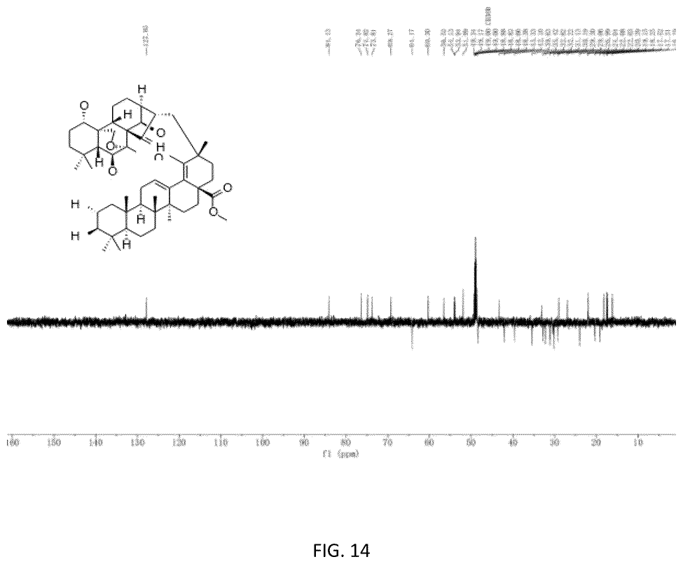

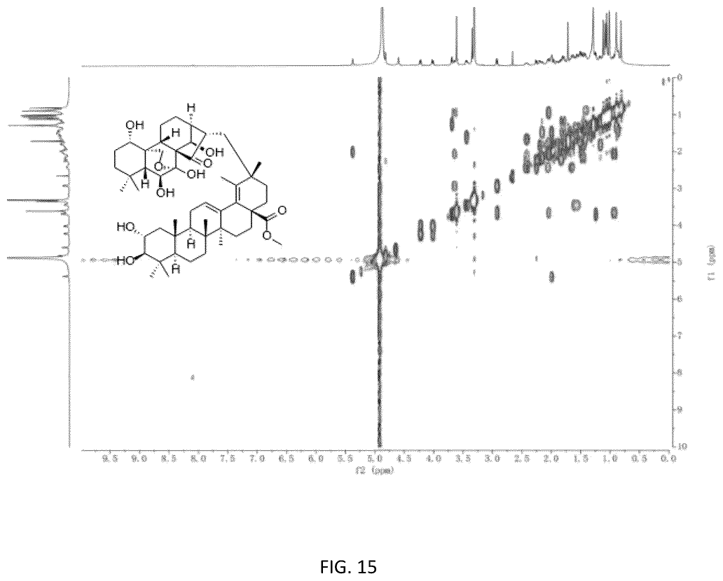

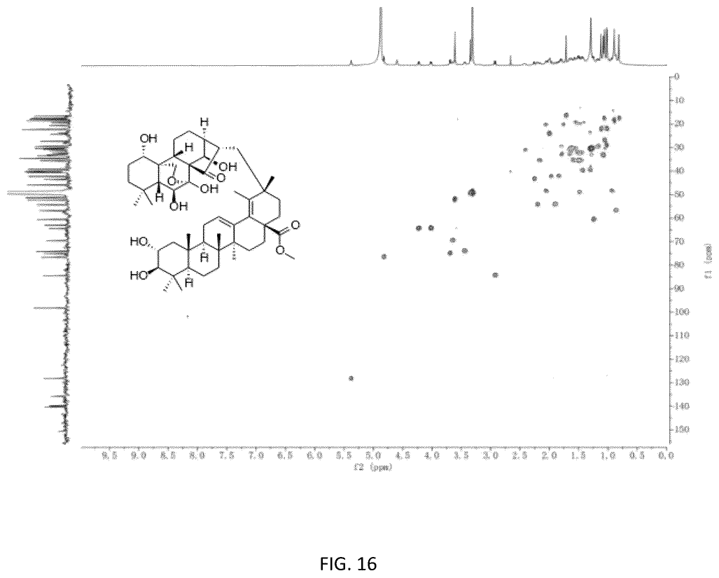

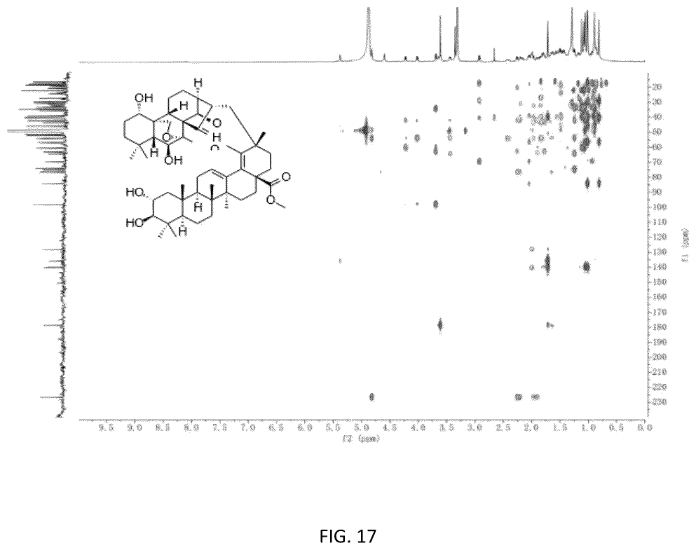

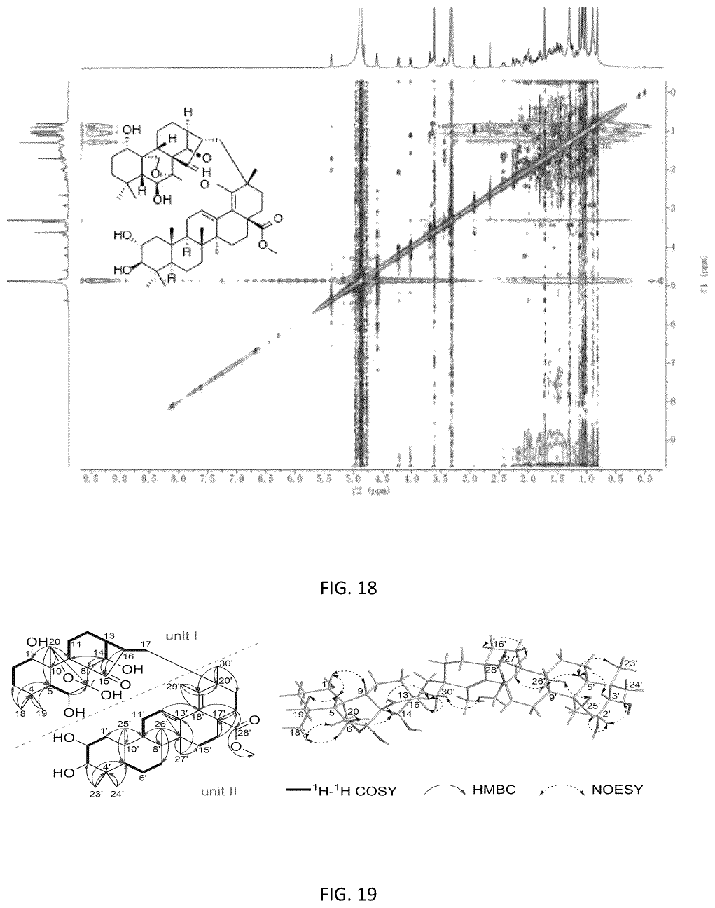

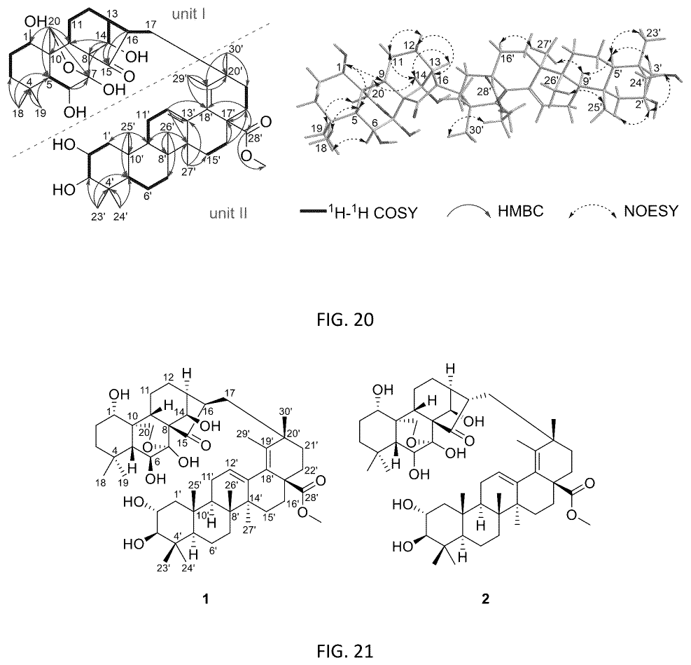

A relative configuration of the compound 1 was determined by analyzing a NOESY spectrum (FIG. 9). Relevant reports have identified H-5 and H-5′ as being β-orientated so far, and related signals in the NOESY proved that CH₃-19/H-58/H-1/H-9 was β-orientated; cross peaks of H-6/CH₃-18/H-20a and H-20b/H-14/H-13/H-16 showed that H-6/H-13/H-14/H-16/CH₃-18 was α-oriented, and an a correlation between H-5′/CH₃-23′ and H-3′/H-5′/H-9′/CH₃-27′/H-16′ showed that H-3′/H-5′/H-9′/CH₃-23′/CH₃-27′ was α-oriented and 28-COOCH₃ was β-oriented; a correlation between 28-COOCH₃ and CH₃-30′ showed that CH₃-30′ was also β-oriented; and it was inferred from a correlation of CH₃-24′/H-2′/CH₃-25′/CH₃-26′ that H-2′, CH₃-24′, CH₃-25′, and CH₃-26′ were 8-oriented. By the measurement and calculation of ECD, it was concluded that an absolute configuration of C-16 was an R configuration, and finally, it was determined that an absolute configuration of the compound 1 was 1S, 5R, 6S, 7S, 8R, 9S, 10S, 13S, 14R, 16R, 2′R, 3′R, 5′R, 8′R, 9′R, 10′R, 14'S, 17'S, and 20'S. Therefore, a complete structure of the compound 1 with the new skeleton was determined, and named Bisicacinol B.

### 3.2 Structural Identification of Bisicacinol C (2)

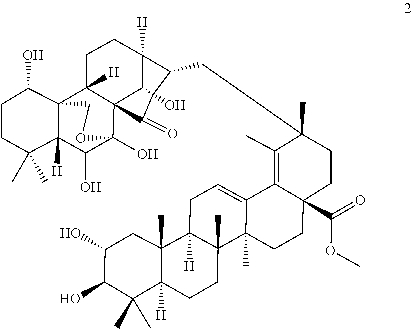

The compound 2 was white amorphous powder, [a]_{D}²⁰+15.0 (c 0.01, MeOH). In combination with ¹³C NMR data and a quasi-molecular ion peak m/z 849.5522 ([M+H]⁺, calculated value of C₅₁H₇₇O₁₀+ of 849.5517) given by a high-resolution mass spectrum (HRESIMS), it could be inferred that a molecular formula of the compound was C₅₁H₇₆O₁₀, and a degree of unsaturation was 14. NMR measurement data of the compound were highly similar to those of the compound 1 (Table 1), and the difference lied in that H-16 (δ_{H} 2.20) and C-16 (δ_{C} 54.4) in the compound 2 were quite different from H-16 (δ_{H} 2.93) and C-16 (δ_{C} 50.1) in the compound 1, and it was inferred that the compound 2 was a 16-epimer of the compound 1. In addition, a coupling constant (J=9.7 Hz, d) of H-16 in the compound 2 was different from that in the compound 1 (J=7.2 Hz t), which also supported the above inference. By the measurement and calculation of ECD, it was determined that C-16 of the compound 2 had an S configuration, and finally, it was determined that an absolute configuration of the compound 2 was 1S, 5R, 6S, 7S, 8R, 9S, 10S, 13S, 14R, 16S, 2′R, 3′R, 5′R, 8′R, 9′R, 10′R, 14'S, 17'S, and 20'S. Therefore, a complete structure of the compound 2 with the new skeleton was determined, and named Bisicacinol C. The compounds 1 and 2 were the first cases of ent-kaurane diterpenoid-ursane triterpenoid heterodimer.

TABLE 1
¹H and ¹³C NMR data of compounds 1
and 2 (¹H, 500 MHz; ¹³C, 125 MHz)

| | 1 | | 2 | |
|---|---|---|---|---|
| position | δ_{H}, (J in Hz) | δ_{C}, type | δ_{H}, (J in Hz) | δ_{C}, type |
| 1 | 3.44, dd (10.5, | 73.7, CH | 3.44, dd (11.5, | 74.1, CH |
| | 6.3) | | 5.6) | |
| 2α | 1.31, | 30.3, CH₂ | 1.57, m | 30.5, CH₂ |
| | overlapped | | | |
| 2β | 1.61, dd (10.4, | | 1.62, m | |
| | 4.2) | | | |
| 3α | 1.31, | 39.8, CH₂ | 1.42, m | 39.9, CH₂ |
| | overlapped | | | |
| 3β | 1.44, | | 1.30, m | |
| | overlapped | | | |
| 4 | | 34.6, C | | 34.6, C |
| 5 | 1.23, d (6.0) | 61.9, CH | 1.25, d (7.2) | 60.6, CH |
| 6 | 3.59, d (5.9) | 74.8, CH | 3.69, d (7.0) | 75.1, CH |
| 7 | | 98.0, C | | 98.2, C |
| 8 | | 49.6, C | | 48.8, C |
| 9 | 1.67, dd (12.1, | 55.1, CH | 1.90, dd | 54.2, CH |
| | 5.2) | | (13.1, 5.5) | |
| 10 | | 42.1, C | | 42.4, C |
| 11α | 2.14, dt (12.9, | 19.9, CH₂ | 2.05, dd | 20.7, CH₂ |
| | 3.5) | | (11.6, 3.7) | |
| 11β | 1.54, m | | 1.75, m | |
| 12α | 1.54, m | 22.0, CH₂ | 2.42, dt (13.7, | 31.4, CH₂ |
| | | | 8.8) | |
| 12β | 2.06, dd (12.6, | | 1.64, m | |
| | 4.7 | | | |
| 13 | 2.55, t (8.6) | 40.9, CH | 2.26, d (9.2) | 43.6, CH |
| 14 | 4.97, d (1.9) | 75.0, CH | 4.82, s | 76.6, CH |
| 15 | | 225.7, C | | 226.4, C |
| 16 | 2.93, d (9.2) | 50.1, CH | 2.20, d (7.2) | 54.4, CH |
| 17α | 2.06, m | 33.7, CH₂ | 1.96, m | 42.3, CH₂ |
| 17β | 1.57, m | | 1.84, d (6.9) | |
| 18 | 1.10, s | 21.8, CH₃ | 1.12, s | 22.3, CH₃ |
| 19 | 1.04, s | 32.9, CH₃ | 1.08, s | 33.4, CH₃ |
| 20a | 4.23, d (10.1) | 64.7, CH₂ | 4.23, d (10.0) | 64.5, CH₂ |
| 20b | 4.03, d (10.3) | | 4.02, d (10.0) | |
| 1′a | 0.94, d (11.9) | 48.7, CH₂ | 0.95, d (12.3) | 48.3, CH₂ |
| 1′β | 2.02, dd (12.6, | | 2.05, dd (11.6, | |
| | 4.7 | | 3.7) | |
| 2′ | 3.64, ddd | 69.6, CH | 3.65, | 69.6, CH |
| | (12.9, 9.7, 4.7) | | overlapped | |
| 3′ | 2.93, d (9.7) | 84.4, CH | 2.92, d (9.5) | 84.4, CH |
| 4′ | | 40.2, C | | 40.2, C |
| 5′ | 0.86, d (11.6) | 56.8, CH | 0.86, | 56.8, CH |
| | | | overlapped | |
| 6′α | 1.54, m | 19.4, CH₂ | 1.53, m | 19.4, CH₂ |
| 6′β | 1.44, m | | 1.42, m | |
| 7′α | 1.51, m | 35.7, CH₂ | 1.57, m | 35.7, CH₂ |
| 7′β | 1.57, m | | 1.48, m | |
| 8′ | | 39.7, C | | 39.1, C |
| 9′ | 1.48, dd (12.0, | 49.4, CH | 1.48, m | 49.2, CH |
| | 3.5) | | | |
| 10′ | | 39.1, C | | 40.5, C |
| 11′α | 1.98, d (3.8) | 24.3, CH₂ | 1.99, dd (8.7, | 24.3, CH₂ |
| | | | 3.8) | |
| 11′β | 2.00, d (3.8) | | 1.29, | |
| | | | overlapped | |
| 12′ | 5.37, t (3.9) | 127.8, CH | 5.38, t (3.9) | 128.1, CH |
| 13′ | | 140.4, C | | 140.2, C |
| 14′ | | 46.0, C | | 45.9, C |
| 15′α | 1.81, td (13.3, | 29.6, CH₂ | 1.79, dd (13.9, | 29.6, CH₂ |
| | 3.7) | | 4.8) | |
| 15′β | 1.14, dt (8.1, | | 1.17, dd (14.1, | |
| | 3.5) | | 4.1) | |
| 16′α | 1.70, m | 32.6, CH₂ | 2.16, dd (13.3, | 35.7, CH₂ |
| | | | 3.5) | |
| 16′β | 1.78, m | | 1.47, m | |
| 17′ | | 51.1, C | | 51.3, C |
| 18′ | | 136.0, C | | 135.7, C |
| 19′ | | 139.1, C | | 139.8, C |
| 20′ | | 40.5, C | | 40.1, C |
| 21′a | 1.56, m | 32.8, CH₂ | 1.79, dd (13.9, | 32.5, CH₂ |
| | | | 4.8) | |
| 21′β | 1.73, m | | 1.64, dd (13.9, | |
| | | | 3.4) | |
| 22′α | 2.14, dt (12.9, | 35.5, CH₂ | 1.47, m | 33.1, CH₂ |
| | 3.5) | | | |
| 22′β | 1.57, m | | 1.52, m | |
| 23′α | 1.02, s | 29.4, CH₃ | 1.01, s | 29.4, CH₃ |
| 24′β | 0.81, s | 17.6, CH₃ | 0.81, s | 17.6, CH₃ |
| 25′ | 1.06, s | 17.8, CH₃ | 1.05, | 17.8, CH₃ |
| | | | overlapped | |
| 26′ | 0.90, s | 18.6, CH₃ | 0.90, s | 18.5, CH₃ |
| 27′ | 0.99, s | 22.5, CH₃ | 1.00, | 22.4, CH₃ |
| | | | overlapped | |
| 28′ | | 178.7, C | | 178.6, C |
| 29′ | 1.73, s | 16.3, CH₃ | 1.72, s | 16.5, CH₃ |
| 30′ | 1.04, s | 27.7, CH₃ | 1.05, | 27.3, CH₃ |
| | | | overlapped | |
| 28′-OCH₃ | 3.62, s | 52.3, CH₃ | 3.61, s | 52.3, CH₃ |

### Embodiment 2

An anti-tumor cell activity test research of the present invention was carried out by the following steps.

### 1. Culture of Tumor Cells

A pancreatic cancer cell line MIA PaCa-2, a colon cancer cell line HT-29, a lung cancer cell line A549, and a liver cancer cell line HepG2 (cell bank of Chinese Academy of Sciences) were cultured in a DMEM culture solution containing 10% fetal bovine serum (Gibco Company) at 37° C. under 5% CO2. HT-29, A549 and HepG2 cell culture media were supplemented with 10% FBS and 1% PSN, and a MIA PaCa-2 cell culture medium was supplemented with 10% FBS, 2.5% HS and 1% PSN.

### 2. Preparation of Experimental Drugs

Proper amounts of the compounds 1 and 2 prepared in the above Embodiment 1 were weighed and dissolved in DMSO, so that mother solutions had a final concentration of 40 mM, and were stored in a refrigerator at 4° C. Before the experiment, the mother solutions were diluted with a DMEM culture medium, so as to make drugs have a concentration of 20 μM and ensure that the DMSO had a final concentration lower than 0.1%. Different volumes of DMEM culture media were added to dilute the compounds into different concentrations. Meanwhile, a DMEM culture medium containing 0.1% DMSO was used as a negative control.

### Toxicities of Drugs to Tumor Cell Lines

Tumor cells were suspended in a culture medium, MIA PaCa-2, HT-29 and HepG2 cells were inoculated into a 96-well plate at a cell density of 8000, A549 cells were inoculated into a 96-well plate at a cell density of 3000 (100 μL/well), and the cells were cultured at 37° C. under 5% CO2 for 24 hours. In a logarithmic growth phase of the tumor cell lines, different concentrations of compounds (0, 0.004, 0.04, 0.4, 4, and 40 μM) were added to culture the cells at 37° C. under 5% CO₂ for 72 hours.

### Detection of Cell Viability by CCK-8 Method

After 72 hours of interaction between the drugs and the tumor cells, 10 μL of CCK-8 solution was added into each well to incubate the cells in a sterile incubator for 1 hour, and then the solution was taken out to determine an OD value by a microplate reader at a wavelength of 450 nm. IC₅₀ (median inhibitory concentration) values of the compounds were calculated by GraphPad Prism 8 software, and 5-FU and Carboplatin were used as positive controls.

Experimental results referred to Table 2.

TABLE 2
Cytotoxic activities of compounds 1 and 2 (IC₅₀: μM)

| | IC₅₀ ± SD (μM)^{a} | | | |
|---|---|---|---|---|
| no. | MIA PaCa-2 | HT-29 | A549 | HepG2 |
| Compound 1 | 13.07 ± 0.18 | 14.95 ± 0.14 | 4.20 ± 0.24 | 14.84 ± 0.12 |
| Compound 2 | >40 | 33.35 ± 0.06 | 23.10 ± 0.12 | >40 |
| 5- | / | 12.87 ± 0.04 | / | 4.82 ± 0.07 |
| Fluorouracil | | | | |
| Carboplatin | / | / | >40 | / |

Experimental conclusion: the evaluation of cytotoxic activities of the compounds 1 and 2 showed that the compound 1 had a strong activity on the pancreatic cancer cell line MIA PaCa-2, the colon cancer cell line HT-29, the lung cancer cell line A549, and the liver cancer cell line HepG2, which was superior to those of the compound 2 and the positive control drugs 5-FU and Carboplatin. The compound 2 also showed a strong inhibitory activity on the colon cancer cell line HT-29 and the lung cancer cell line A549, which was superior to those of the positive drugs 5-FU and Carboplatin, so that the compound had the potential to develop a new anti-lung cancer drug.

### Embodiment 3

An antibacterial activity test research of the present invention was carried out by the following steps.

### 1. Experimental Materials and Reagents

Helicobacter pylori (G27 and HP129) and Candida albicans (SC5314 and C5) both came from Professor Hongkai Bi's laboratory of Nanjing Medical University. Main culture mediums and main reagents: Colombian culture medium, LB culture medium, selective antibiotics (metronidazole and amphotericin B), serum, and the like.

### 2. Preparation of Culture Media

Colombian liquid culture medium: 29.0 g of Colombian liquid culture medium was accurately weighed, heated, and dissolved in 1000 mL of double distilled water, and the culture medium was subjected to autoclaved sterilization at 121° C. for 15 minutes after being completely dissolved for later use.

Colombian blood agar solid culture medium: 39.0 g of Colombian blood agar solid culture medium was accurately weighed, heated, and dissolved in 1000 mL of double distilled water, and the culture medium was subjected to autoclaved sterilization at 121° C. for 15 minutes after being completely dissolved, naturally cooled to about 50° C. after the autoclaved sterilization, quickly added with 5% sterile deflbrinated sheep blood, mixed evenly, and then poured into a sterile dish while the mixture was hot.

LB culture medium: 15 g/L glucose, 10 g/L yeast extract, 5 g/L peptone, and 10 g/L sodium chloride.

### 3. Resuscitation and Culture of Tested Strains

Standard strains of Helicobacter pylori were taken out of a refrigerator at −80° C. and placed at room temperature, 200 μL of standard strains were accurately sucked, transferred to a solid culture medium, and spread with an L-shaped glass rod, a culture dish loaded with the strains was put into a culture bag, then an AnaeroPack was put into the culture bag, and the culture bag was quickly sealed and then put into a constant-temperature incubator at 37° C. to culture the strains for 72 hours. After the end of culture, the Helicobacter pylori was identified first, then a bacterial lawn on the solid culture medium was scraped off, and the bacterial lawn was transferred into 50 mL of liquid culture medium to serve as an original bacterial solution.

### 4. Determination of MIC

(1) Sample solutions of the compounds 1 and 2 prepared in Embodiment 1 were configured to have a concentration of 2 mg/mL.

(2) In preparation by a MIC plate, a first well was added with 173.6 μL of culture medium and then added with 6.4 μL of antibacterial drug, and dilution was carried out by multiple proportions to a 7^{th} well; and an 8^{th} well was not added with the drug, but was reserved with 90 μL of culture medium to serve as a control with bacteria but no drug.

(3) In preparation of bacterial solution, the Helicobacter pylori growing at a logarithmic phase on a solid plate was made into a bacterial suspension with a BHI culture medium, an OD600 value of concentration of the bacterial suspension was adjusted to be 0.3 (1×10⁸ CFU/mL), and then the bacterial suspension was diluted 10 times to reach 1×10⁷ CFU/mL for later use.

(4) In inoculation, 10 μL of bacterial solution was added into the 1st to 8^{th} wells (a concentration of the bacterial solution in each well was about 1.0×10⁶ CFU/mL). Results were judged after 72 hours of culture. Drug concentrations from the 1st to 7^{th} wells were 64 μg/mL, 32 μg/mL, 16 μg/mL, 8 μg/mL, 4 μg/mL, 2 μg/mL, and 1 μg/mL respectively.

(5) In result judgment, a lowest drug concentration capable of completely inhibiting the growth of bacteria in the wells was MIC. The test was only meaningful when the bacteria in the 8^{th} well of the positive control well (without antibiotics) grew obviously. When there was a single drift in a microdilution method, a highest drug concentration capable of inhibiting the growth of bacteria should be recorded. If there were many drifts, the results should not be reported, and the test should be repeated. Each drug was tested repeatedly for 3 times.

### 5. Experimental Results Referred to Tables 3 and 4.

TABLE 3
Inhibitory effects of compounds 1 and
2 on different strains (MIC: μg/ml)

| Strain | 1 | 2 | MTZ |
|---|---|---|---|
| Helicobacter pylori H. pylori G27 | 16 | 16 | 2 |
| Helicobacter pylori H. pylori 129 | 32 | 32 | 8 (R) |

MTZ, metronidazole;
R, drug-resistant strain

TABLE 4
Synergistic inhibitory effect of compound 1 and amphotericin
B (AMB) on different strains (MIC: μg/ml)

| | Compound 1 | | | | MIC | | |
|---|---|---|---|---|---|---|---|
| | (μg/ml) | | AMB (μg/ml) | | (μg/ml) + | FICI index | |
| | Separate | Combined | Separate | Combined | 0.5 μg/ml | after | Mode of |
| Strain | use | use | use | use | AMB | combination | action |
| SC5314 | >32 | 1 | 2 | ≤0.25 | 1 | ≤0.078 | Synergistic |
| | | | | | | | effect |
| C5 | >32 | 1 | 2 | ≤0.25 | 1 | ≤0.078 | Synergistic |
| | | | | | | | effect |

The above experimental results show that the compounds 1 and 2 have a good antibacterial activity on both sensitive Helicobacter pylori and drug-resistant Helicobacter pylori, and the MIC is 16-32 μg/mL, which indicates that the compounds 1 and 2 have an inhibitory effect on Helicobacter pylori. Compound 1 has no significant inhibitory effect on the growth of two kinds of Candida albicans when used separately, but when the compound is used in combination with AMB, the MIC of the compound can be greatly reduced, and the MIC of AMB can be reduced below 0.25 μg/mL at the same time, so that the compound can be used to prepare an anti-Candida albicans drug or serve as a lead compound for the development of anti-Candida albicans drug.

What is claimed is:

1. A compound with a new skeleton of ursane triterpenoid—ent-kaurane diterpenoid dimer, comprising compounds 1 and 2 as shown by the following structural formulae:

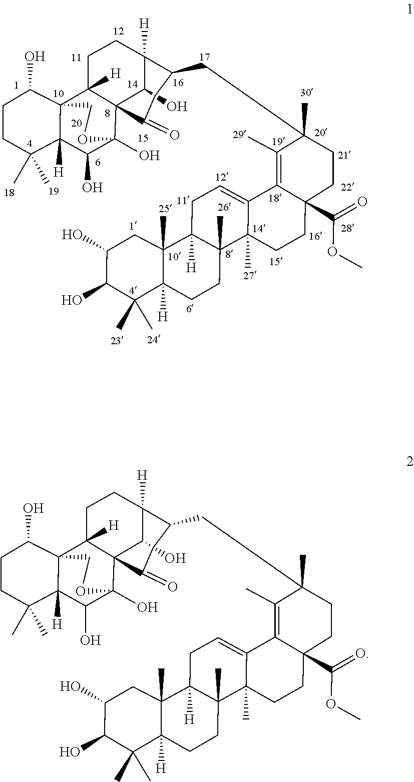

2. The compound according to claim 1, wherein the compound and a pharmaceutically acceptable carrier are prepared into a pharmaceutical drug.

3. The compound according to claim 1, wherein the pharmaceutical drug is mixed with an amphotericin B as an anti-Candida albicans drug.

4. A preparation method of the compound according to claim 1, comprising the following steps:
(1) weighing and crushing dried tuber of I. trichantha, adding ethanol aqueous solution, refluxing the mixture for extraction of medicinal material, filtering the mixture and then collecting a filtrate, and concentrating the filtrate under a reduced pressure until no ethanol smell exists to obtain a concentrated solution;
(2) extracting the concentrated solution obtained in the step (1) with petroleum ether, dichloromethane, ethyl acetate, and n-butanol respectively, and concentrating the extracts under a reduced pressure to obtain a petroleum ether fraction, a dichloromethane fraction, an ethyl acetate fraction, an n-butanol fraction, and a raffinate fraction respectively;
(3) subjecting the dichloromethane fraction obtained in the step (2) to AB-8 macroporous resin atmospheric column chromatography, and eluting the product with ethanol aqueous solution to obtain 3 sub-fractions DCM-1 to DCM-3;
(4) subjecting the eluate fraction DCM-3 obtained in the step (3) to silica gel atmospheric column chromatography, and eluting the product with dichloromethane-methanol to obtain 7 secondary fractions DCM-3-1 to DCM-3-7; and
(5) subjecting the fraction DCM-3-5 obtained in the step (4) to medium-pressure preparative—MCI column chromatography, and gradiently eluting the product with methanol aqueous solution serving as a mobile phase to obtain 11 fractions DCM-3-5-1 to DCM-3-5-11; and subjecting the fraction DCM-3-5-9 to semi-preparative high-performance liquid chromatography to obtain the compounds 1 and 2.

5. The preparation method of the compound according to claim 2, comprising the following steps:
(1) weighing and crushing dried tuber of I. trichantha, adding ethanol at a volume concentration of 80-95%, refluxing the mixture for extraction of medicinal material according to a material-liquid ratio of 1:6-20 for 1-3 times, for 1-3 hours each time, filtering the mixture and then collecting filtrates, combining the filtrates, and concentrating the combined filtrate under a reduced pressure until no ethanol smell exists to obtain an extractum;
(2) preparing the extractum obtained in the step (1) into a suspension with a proper amount of water, and then extracting the suspension with petroleum ether, dichloromethane, ethyl acetate, and n-butanol at an equal volume for 3-6 times respectively to obtain a petroleum ether fraction, a dichloromethane fraction, an ethyl acetate fraction, an n-butanol fraction, and a raffinate fraction respectively;
(3) subjecting the dichloromethane fraction obtained in the step (2) to macroporous resin sample stirring and then to macroporous resin atmospheric column chromatography, and gradiently eluting the product with ethanol aqueous solution serving as a mobile phase at volume ratios of 30:70, 60:40, 90:10, and 100:0 in sequence according to 4-6 column volumes in each gradient elution; and after thin-layer and ultra-high-performance liquid chromatography, concentrating and combining the products by a rotary evaporator to obtain 3 sub-fractions DCM-1 to DCM-3;
(4) subjecting the eluate fraction DCM-3 obtained in the step (3) to silica gel sample stirring and then to silica gel atmospheric column chromatography, and gradiently eluting the product with dichloromethane-methanol serving as a mobile phase at volume ratios of 100:0, 50:1, 30:1, 20:1, 10:1, 7:1, 5:1, 3:1, 2:1, 1:1, 1:3, 1:7, and 0:100 in sequence according to 3-6 column volumes in each gradient elution; and after thin-layer and ultra-high-performance liquid chromatography, concentrating and combining the products by a rotary evaporator to obtain 7 sub-fractions DCM-3-1 to DCM-3-7;
(5) subjecting the fraction DCM-3-5 obtained in the step (4) to MCI sample stirring and then to medium-pressure preparative-MCI column chromatography, and gradiently eluting the product with methanol aqueous solution serving as a mobile phase, wherein A is water, and B is methanol; and a gradient elution program is as follows: 0.01-20.00 minutes, 30%-30% B; 20.00-50.00 minutes, 40%-40% B; 50.00-80.00 minutes, 45%-45% B; 80.00-110.00 minutes, 50%-50% B; 110.00-140.00 minutes, 55%-55% B; 140.00-170.00 minutes, 60%-60% B; 170.00-200.00 minutes, 70%-70% B; 200.00-230.00 minutes, 85%-85% B; 230.00-270.00 minutes, 100%-100% B, an elution flow rate of 20 mL/min, and detection wavelengths of 256 nm and 310 nm; and after ultra-high-performance liquid chromatography, concentrating and combining the products to obtain 11 fractions DCM-3-5-1 to DCM-3-5-11; and
(6) subjecting the eluate fraction DCM-3-5-9 of 200-220 minutes obtained in the step (5) to semi-preparative high-performance liquid chromatography, isocratically eluting the product with pure water A-methanol B serving as a mobile phase, and separating the eluate to obtain the compound 1 and the compound 2.

6. The preparation method of the compound according to claim 3, wherein chromatographic conditions of semi-preparative high-performance liquid chromatography are as follows: a chromatographic column model of Hedera ODS preparative chromatographic column, specifications of 10 nm, 5 μm, and 10×250 mm, a column pressure of high-performance liquid chromatography of 9.8 MPa, a column temperature of 22-26° C., an injection volume of 100 μL, a mobile phase of MeOH and H₂O at a volume ratio of 80:20, a flow rate of 3 mL/min, and a detection wavelength of 310 nm.

7. A method for treating a disease comprising a step of administering the compound according to claim 1 to a subject in need, wherein the disease is a cancer or an infective disease.

8. The method according claim 7, wherein the cancer is selected from the group consisting of a pancreatic cancer, a colon cancer, a lung cancer and a liver cancer.

9. The method according claim 7, wherein the infective disease is caused from a bacterial or a fungal.

10. The method according claim 9, wherein the bacteria is Helicobacter pylori.

11. The method according claim 9, wherein the fungal is Candida albicans.
